(19) United States
(12) Patent Application Publication
(10) Pub. No.: US 2023/0172753 A1
(43) Pub. Date: Jun. 8, 2023

(54) METHOD FOR TREATING ADVANCED PRIMARY ANGLE-CLOSURE GLAUCOMA WITH CATARACT

(71) Applicant: Zhongshan Ophthalmic Center of Sun Yat-sen University, Guangzhou (CN)

(72) Inventors: Xiulan ZHANG, Guangzhou (CN); Yunhe SONG, Guangzhou (CN); Shida CHEN, Guangzhou (CN); Xinbo GAO, Guangzhou (CN); Fengbin LIN, Guangzhou (CN)

(21) Appl. No.: 17/544,275

(22) Filed: Dec. 7, 2021

(51) Int. Cl. A61F 9/007; A61F 9/00; G02B 21/00

(52) U.S. Cl. CPC A61F 9/00781; A61F 9/00754; A61F 9/0008; G02B 21/0012

(57) ABSTRACT

A method for treating advanced primary angle-closure glaucoma (PACG) with cataract includes the steps of: S1, utilizing phacoemulsification cataract extraction combined with intraocular lens implantation (PEI) to remove pupillary block and shallow anterior chamber and reduce a risk of progressive formation of peripheral anterior synechia (PAS); S2, utilizing goinosynchialysis (GSL) to separate the physical adhesion of PAS in the anterior chamber angle with the assistance of a gonioscope and a chopper; and S3, utilizing goniotomy (GT) to incise a dysfunctional trabecular meshwork and a Schlemm canal, so as to remove a dysfunctional trabecular meshwork, enhance outflow of aqueous humor, and reduce intraocular pressure.

### TECHNICAL FIELD

The present invention relates to the technical field of ophthalmic surgery, and in particular to a method for treating advanced primary angle-closure glaucoma with cataract.

### BACKGROUND ART

The primary angle-closure glaucoma (PACG) is a common disease in China, which has a high prevalence rate and blindness rate. It is reported that there are half of global PACG cases in China. PACG will cause closure of the anterior chamber angle, outflow obstruction of aqueous humor and increase of intraocular pressure. The increase of outflow resistance of aqueous humor caused by pupillary block, progressive closure of the anterior chamber angle and pathological changes in the trabecular meshwork and the Schlemm canal mainly renders the increase of intraocular pressure caused by the PACG.

Surgery is mainly used for treating the advanced PACG, which includes trabeculectomy and combined glaucoma-cataract surgery. The combined glaucoma-cataract surgery is phacoemulsification cataract extraction combined with intraocular lens implantation (PEI) combined with trabeculectomy. Trabeculectomy is a traditional anti-glaucoma surgical method, of which the principle is to manufacture an aqueous humor filtration channel such that aqueous humor can flow out from this channel, thereby achieving the purposes of reducing intraocular pressure and treating glaucoma. However, trabeculectomy is not perfect due to (1) numerous complications, such as shallow anterior chamber, hyphema, persistent low intraocular pressure, decompensation of corneal endothelium, endophthalmitis and bleb-related complications; (2) large trauma, damage to the conjunctiva and sclera by incision on the eyeball wall, and long time for recovery from wound; (3) long surgery time with at least 40-50 minutes required by the traditional combined glaucoma-cataract surgery; (4) long learning curve for doctors; and (5) precisely postoperative care and other technical trouble.

So far, it has been proved that PEI alone or PEI combined with goniosynechialysis (GSL) can effectively treat PACG in terms of reducing the intraocular pressure of the angle-closure glaucoma and reducing the use of antiglaucoma medications. However, some researches have shown that there is no statistical difference between PEI alone and PEI+GSL in the effect of treating the PACG, and it cannot be proved that PEI+GSL can improve the function of additionally reducing intraocular pressure in PACG patients compared with PEI alone. PEI+GSL is obviously insufficient in the treatment of the advanced PACG.

Minimally invasive glaucoma surgeries (MIGS) in development have made a breakthrough in the treatment of glaucoma, and are proved to be safe and effective in open-angle glaucoma. So far, Schlemm canal incision, also known as goniotomy (GT), is the most common and effective in the MIGS, which is featured in relatively few complications, minimally invasive, rapid recovery, capability to effectively reduce intraocular pressure and relatively simple operation. However, there is still a lack of minimally invasive surgery-based normative idea and method for treating advanced PACG.

### SUMMARY

The present invention thus provides a method for treating advanced PACG with cataract, which can effectively treat the disease, and simultaneously solve the technical disadvantages of traditional surgery.

According to present invention, the method for treating advanced PACG with cataract includes the steps of:

S1, utilizing phacoemulsification cataract extraction combined with intraocular lens implantation (PET) to remove pupillary block and shallow anterior chamber and reduce a risk of progressive formation of peripheral anterior synechia (PAS);

S2, utilizing goinosynchialysis (GSL) to separate anterior chamber angle with the assistance of a gonioscope and a chopper, so as to remove physical adhesion of the anterior chamber angle; and

S3, utilizing goniotomy (GT) to incise trabecular meshwork and open Schlemm canal, so as to remove a dysfunctional trabecular meshwork, enhance outflow of aqueous humor, and reduce intraocular pressure.

In step of S1, the PEI mainly solves the problems of pupillary block and shallow anterior chamber of the PACG. In step of S2, the GSL mainly solves the problem of adhesion of the anterior chamber angle of the PACG. In step of S3, the GT mainly solves the problem that lesion of the trabecular meshwork and the Schlemm canal obstructs outflow of aqueous humor in the PACG. By means of the PEI, the problem of the shallow anterior chamber is solved, the pupillary block is relived, the anterior chamber is deepened to enhance outflow of the trabecular meshwork, and the risk of progressive formation of the PAS is reduced. The GSL is carried out with the assistance of the gonioscope and a chopper, thereby mechanically separating and opening the anterior chamber angle anew. By means of the GT, the dysfunctional trabecular meshwork is removed, and the Schlemm canal is opened to enhance outflow of the aqueous humor, thereby finally achieving intraocular pressure reduction and treating the PACG.

Note: (1) The shallow anterior chamber is the most common complication in an early stage after glaucoma filtration surgery, which commonly takes place with too strong filtration and is mostly caused by improper sclera flap manufacturing, too large sclera fistula orifice, poor valve tightness and too large filtering bleb;

(2) The Schlemm canal is a tube located within the eye, and is also known as a sinus venous sclerae or Schlemm's canal.

According to the present invention, step of S1 includes:

S11, carrying out conventional disinfection and surface anesthesia on a surgical eye;

S12, using an eye speculum to open eyelids, making a main corneal incision with a length of 1.8-3.2 mm at an upper quadrant or temporal quadrant, and making a side port cornea incision in a direction with 90° relative to the main incision;

S13, injecting a viscoelastic substance into an anterior chamber;

S14, carrying out continuous curvilinear capsularhexis for an area of about 5.5 mm*5.5 mm, and using a balanced salt solution to carry out hydrodissection and phacoemulsification to suck out a nucleus lentis, where epinephrine may be added into the balanced salt solution as required;

S15, sucking, by irrigation and aspiration, residual cortex lentis; and

S16, implanting an intraocular lens into posterior capsule

Specifically, there are pupillary block and shallow anterior chamber in the PACG, by means of PEI, the anterior chamber is deepened to enhance outflow of the trabecular meshwork, and the risk of progressive formation of PAS is reduced, thereby achieving the purpose of reducing resistance to outflow of the aqueous humor.

According to the present invention, step of S2 includes:

S21, injecting a viscoelastic substance into an anterior chamber, and coating a surface of a cornea with the viscoelastic substance; and

S22, using a chopper with the assistance of the gonioscope, inserting a contralateral anterior chamber angle from the main incision and slightly pressing the root of an iris, so as to separate the adhesive anterior chamber angle by 120° until a lower ⅔ functional trabecular meshwork and a scleral spur are seen.

Specifically, in step S22, a head position of a patient and an inclination angle of a surgical microscope are adjusted to clearly and directly view an anterior chamber angle structure under the gonioscope.

Specifically, the progressive PAS finally causes closure of the anterior chamber angle and increased intraocular pressure, resulting in irreversible optic nerve damage. The GSL with the assistance of the gonioscope and the chopper may effectively remove physical adhesion of the anterior chamber angle, and if a function of a separated exposed trabecula is intact and an outflow passage behind the trabecula is kept perfectly, the outflow resistance of the aqueous humor may be further reduced.

According to the present invention, step S3 includes the steps:

S31, goniotomy (GT) is performed using microhook or microblade for goniotomy to incise trabecular meshwork and the inner wall of Schlemm canal with a range of 120 degrees.

S32, carrying out, by irrigation and aspiration, suction to remove the viscoelastic substance and possible hyphema in an anterior chamber; and

S33, forming the anterior chamber using a balanced salt solution, and suturing the cornea incision in a watertight manner, where epinephrine may be added into the balanced salt solution as required.

Specifically, there are also pathological changes in the trabecular meshwork and the Schlemm canal of the PACG, for example, early progressive trabecular fibrosis of the PACG, narrowing or disappearance of a trabecular meshwork, the decreased number of endothelial cells, and pigment particles contained in part of endothelial cell cytoplasm; and obstruction of outflow of the aqueous humor caused by fibrotic degeneration of most of advanced trabecular meshwork, pigmentation, disappearance of a mesh structure and narrow or occluded Schlemm canal. The GT with the assistance of the gonioscope and the microhook or microblade for goniotomy may incise the lesion trabecular meshwork and Schlemm canal tissue, thereby further reducing the outflow resistance of the aqueous humor, and further increasing drainage of the aqueous humor through an internal passage.

The main incision is sutured with a 10-0 nylon suture if needed after step S3 is carried out.

According to the present invention, the method further includes step S4, utilizing an eye pad to bind the surgical eye with tobramycin and dexamethasone ophthalmic ointment, and 1% pilocarpine ophthalmic ointment.

Compared with the prior art, the present invention has the beneficial effects:

The present invention constructs, by innovatively combining a minimally invasive surgery technology, a set of standard minimally invasive surgery treatment method for advanced PACG with cataract, and further has the effects of less trauma, fewer complications, shortened surgery time and significantly shortened learning curves for doctors while achieving a treatment effect similar to that of traditional surgery.

Surgical operation of the present invention is normalized and standardized, which can effectively solve the problems of sclera flap manufacturing, use of antimetabolite drugs, non-normalized operation, etc. of traditional filtration surgery.

The method of the present invention has small trauma and short surgical time. The surgery may be completed only by means of main corneal incision with a length of 1.8-3.2 mm and the side port incision with a length of 0.5-1.0 mm. That is, all operations may be completed only by means of the surgical incision of the PEI, and compared with large-range conjunctiva, cornea and sclera damage of the traditional surgery technology, the operation may greatly reduce trauma. Operation time of the surgery according to the present invention is 15-20 minutes, while traditional filtration surgery requires 40-60 minutes.

Complications related to a filtering bleb occurring in the traditional surgery are important reasons for surgery failure. However, the present invention does not generate dependence of the filtration bleb of the traditional filtration channel surgery, thereby reducing post-surgical complications.

What's more, the present invention has a short learning curve. Trained doctors may fully grasp the use of the surgical method within a short time.

### DETAILED DESCRIPTION

The embodiment discloses a method for treating advanced PACG with cataract. The method includes the steps of:

S1, utilize phacoemulsification cataract extraction combined with intraocular lens implantation (PEI).

The step of S1 specifically includes:

S11, carrying out conventional disinfection and surface anesthesia on a surgical eye;

S12, using an eye speculum to open eyelids, making a main corneal incision with a length of 1.8-3.2 mm at the upper or temporal quadrant and make side port cornea incision in a direction with 90° relative to the main incision;

S13, injecting a viscoelastic substance into an anterior chamber;

S14, carrying out continuous curvilinear capsularhexis for an area of about 5.5 mm*5.5 mm, and using a balanced salt solution to carry out hydrodissection and phacoemulsification to suck out a nucleus lentis, where epinephrine may be added into the balanced salt solution as required;

S15, sucking, by irrigation and aspiration, residual cortex lentis; and

S16, implanting a posterior chamber intraocular lens.

S2, utilizing goinosynchialysis (GSL) to separate the PAS with the assistance of a gonioscope and a chopper. The step of S2 specifically includes:

S21, injecting a viscoelastic substance into an anterior chamber, and coating a surface of a cornea with the viscoelastic substance; and

S22, adjusting a head position of a patient and an inclination angle of a surgical microscope with the assistance of the gonioscope, so as to clearly and directly view an anterior chamber angle structure under the gonioscope; and the chopper inserting a contralateral anterior chamber angle from the main incision and slightly pressing a root of an iris, so as to separate the PAS in the adhesive anterior chamber angle by 120° until a lower ⅔ functional trabecular meshwork and a scleral spur are seen.

S3, utilizing Goniotomy (GT) to incise dysfunctional trabecular meshwork and a Schlemm canal. The step of S3 specifically includes:

S31, inserting the microhook or microblade for goniotomy into the trabecular meshwork and inserting the Schlemm canal to incise the inner wall of the Schlemm canal by about 120°;

S32, carrying out, by irrigation and aspiration, suction to remove the viscoelastic substance and possible hyphema in an anterior chamber; and

S33, forming an anterior chamber from a balanced salt solution, and suturing the cornea incision in a watertight manner, where epinephrine may be added into the balanced salt solution as required.

In necessity, the main incision may be sutured with a 10-0 nylon suture optionally. Finally, an eye pad is utilized to bind the surgical eye with tobramycin and dexamethasone ophthalmic ointment, and 1% pilocarpine ophthalmic ointment.

In step of S1, the PEI mainly solves the problems of pupillary block and shallow anterior chamber of the PACG with cataract. Specifically, by means of PEI, the anterior chamber is deepened to enhance outflow of the trabecular meshwork, so that the risk of formation of the progressive PAS is reduced, thereby achieving the purpose of reducing resistance to outflow of the aqueous humor.

In step of S2, the GSL mainly solves the problem of adhesion of the anterior chamber angle of the PACG. Specifically, the progressive PAS finally causes closure of the anterior chamber angle and increased intraocular pressure, resulting in irreversible optic nerve damage. The GSL with the assistance of the gonioscope and the chopper may effectively remove physical adhesion of the anterior chamber angle, and the GSL is carried out with the assistance of the gonioscope and a chopper, thereby mechanically separating and opening the anterior chamber angle anew. If a function of a separated exposed trabecula is intact and an outflow passage behind the trabecula is kept perfectly, the outflow resistance of the aqueous humor may be further reduced.

In step of S3, the GT mainly solves the problem that lesion of the trabecular meshwork and the Schlemm canal obstructs outflow of aqueous humor in the PACG. Specifically, there are also pathological changes in the trabecular meshwork and the Schlemm canal of the PACG, for example, early progressive trabecular fibrosis of the PACG, narrowing or disappearance of a trabecular meshwork, the decreased number of endothelial cells, and pigment particles contained in part of endothelial cell cytoplasm; and obstruction of outflow of the aqueous humor caused by fibrotic degeneration of most of advanced trabeculae, pigmentation, disappearance of a mesh structure and narrow or occluded Schlemm canal. The GT with the assistance of the gonioscope and the microhook or microblade for goniotomy may incise the lesion trabecular meshwork and Schlemm canal tissue. By means of the GT, the dysfunctional trabecular meshwork loss is removed, and the Schlemm canal is opened to enhance outflow of the aqueous humor, thereby further reducing the outflow resistance of the aqueous humor, further increasing drainage of the aqueous humor through an internal passage, and finally achieving the purposes of reducing intraocular pressure and treating the PACG.

Obviously, the above embodiment is merely an example given for clearly illustrating the technical solution of the present invention, and is not intended to limit the specific embodiment of the present invention. Any modifications, equivalent replacements, improvements, etc. made within the spirit and principle of claims of the present invention should all fall within the scope of protection of claims of the present invention.

1. A method for treating advanced PACG with cataract, comprising steps of:
S1, utilizing phacoemulsification cataract extraction combined with intraocular lens implantation, so as to remove pupillary block and shallow anterior chamber, and reduce a risk of progressive formation of peripheral anterior synechia;
S2, utilizing goniosynechialysis to separate anterior chamber angle, with the assistance of a gonioscope and a chopper, so as to remove physical adhesion of the anterior chamber angle; and
S3, utilizing goniotomy to incise a diseased trabecular meshwork and Schlemm canal, so as to remove the trabecular meshwork with function loss, enhance outflow of aqueous humor, and reduce intraocular pressure.

2. The method according to claim 1, wherein step S1 comprises steps of:
S11, carrying out conventional disinfection and surface anesthesia on a surgical eye;
S12, using an eye speculum to open eyelids, making a main corneal incision with a length of 1.8-3.2 mm at an upper or temporal quadrant and making a side port cornea incision in a direction with 90° formed between same and the main corneal incision;
S13, injecting a viscoelastic substance into an anterior chamber;
S14, carrying out continuous curvilinear capsularhexis for an area of 5.5 mm*5.5 mm, and using a balanced salt solution to carry out hydrodissection and phacoemulsification to suck out a nucleus lentis;
S15, sucking, by irrigation and aspiration, residual cortex lentis; and
S16, implanting a posterior chamber intraocular lens.

3. The method according to claim 1, wherein step S2 comprises steps of:
S21, injecting a viscoelastic substance into an anterior chamber, and coating a surface of a cornea with the viscoelastic substance; and
S22, using a chopper with the assistance of the gonioscope, inserting a contralateral anterior chamber angle from the main incision and slightly pressing the root of an iris, so as to separate the adhesive anterior chamber angle by 120° until a lower ⅔ functional trabecular meshwork and a scleral spur are seen.

4. The method according to claim 3, wherein in the step of S22, a head position of a patient and an inclination angle of a surgical microscope are adjusted to clearly and directly view an anterior chamber angle structure under the gonioscope.

5. The method according to claim 1, wherein S3 comprises steps of:
S31, inserting a microhook or microblade for goniotomy into the trabecular meshwork and entering the Schlemm canal to incise an inner wall of the Schlemm canal by 120°;
S32, carrying out, by irrigation and aspiration, suction to remove a viscoelastic substance and possible hyphema in an anterior chamber; and
S33, forming an anterior chamber from a balanced salt solution, and suturing a cornea incision in a watertight manner.

6. The method according to claim 1, wherein a main incision is sutured with a 10-0 nylon suture optionally after step S3 is carried out.

7. The method according to claim 1, further comprising step S4, utilizing an eye pad to bind a surgical eye with tobramycin and dexamethasone ophthalmic ointment, and 1% pilocarpine ophthalmic ointment.
